(19) United States
(12) Patent Application Publication
(10) Pub. No.: US 2020/0360196 A9
(43) Pub. Date: Nov. 19, 2020

(54) Diapers with wipes

(71) Applicants: Marva Joy Kelly, Orlando, FL (US); Dianna Lureen Kelly, Silver Spring, MD (US); Lorna Lilieth Kelly, Orlando, FL (US); Marjorie Delarene Kelly, Orlando, FL (US)

(72) Inventors: Marva Joy Kelly, Orlando, FL (US); Dianna Lureen Kelly, Silver Spring, MD (US); Lorna Lilieth Kelly, Orlando, FL (US); Marjorie Delarene Kelly, Orlando, FL (US)

(21) Appl. No.: 16/350,306

(22) Filed: Oct. 31, 2018

Related U.S. Application Data
(60) Provisional application No. 62/707,391, filed on Nov. 1, 2017.

(51) Int. Cl. A61F 13/496; A61K 8/02

(52) U.S. Cl. CPC A61F 13/496; A47K 2010/3266; A61K 8/0208

(57) ABSTRACT

The Present invention comprises of a packet filled with wet wipes configured on the front or back of the diaper. The placement device make it convenient to change a diaper. The diapers would have wet wipes in a removable foil packet, it would be waterproof to keep the moisture from drying out, the packet contains four or five wipes and ready for immediate use.

### RELATED APPLICATIONS

The present invention was first described in and claims of the provisional application No. 62/700,391 Field, Nov. 1, 2017 the entire disclosure of which are incorporated herein by Reference.

### FIELD OF THE INVENTION

The present invention relates to a modified diaper that includes a packet filled with wet wipes on the front or back of the diaper. The packet is waterproof to keep the wipes from drying out each packet contain four to five wipes.

### BACKGROUND OF THE INVENTION

Parent with infant and young children have been using diapers for many years they are also used by caregivers and adults who have problems with incontinence and overactive bladder. Diapers with Wipes would address these problems that arises when using a diaper, when such leakage accident occur which can be messy and time consuming to clean up, having access to Diapers with Wipes immediately would make it easier to address the problem.

There would be no need to search for a container of wipes. Diapers with Wipes would insure that needed supplies are positioned within reach and everything needed for a diaper change would be obtainable when needed. The user may remove the packet of wet wipes and proceed with changing the child's diaper and utilizing the wipes to clean. When the wet wipes are finish user may place the used wipes back in the packet and discard in trash.

The present invention would be beneficial to many parent and caregivers they would save time and effort when changing an infant. Diapers with Wipes would reduce the need to carry a container of wipe. It would be particularity appealing in its ability to lighten the load of a heavy baby bag.

There exist a need to make it easier to change a diaper and provide added peace of mind for parents and caregivers.

The drawback of the prior art is that parents and caregivers would have to carry a container of Wipes every time they need to change a diaper. The frustration comes from running out of wipes when There is a sudden case of loose stool or diarrhea and there's no wipes left in the container to clean up the feces, this can be very stressful for parents and caregivers.

The present invention would reduce and eliminate the stress of changing a diaper, the diaper would be equip with wet wipes for a diaper change. The preferred embodiment of the present invention Provides a means relating to the prior art. The embodiment thereof it will be understood by those skilled in the art that the present invention may be perform without difficulty or effort.

### BRIEF SUMMARY OF THE INVENTION

The present invention comprises of a packet filled with wet wipes configured on the front or back of the diaper. The placement device would make it convenient to change a diaper. The diaper would have wet wipes in a removable foil packet it would be waterproof to keep the moisture from drying out and ready for immediate use.

### BRIEF DESCRIPTION OF THE DRAWING

The present advantage of the invention is illustrated with detailed description and claims and are not limited by the figures of the accompanying drawing in which like references may indicate similar elements and in which:

FIG. 1 is a front view of an open diaper 20, a packet of wet wipes 30, for a diaper according to a preferred embodiment of the present invention.

FIG. 2 is a back view of an open diaper 20, a packet of wet wipes 30, for a diaper according to a preferred embodiment of the present invention.

FIG. 3 is a perspective view of a close diaper 20, the wet wipes 30, for a diaper according to a preferred embodiment of the present invention.

### DETAILED DESCRIPTION OF THE INVENTION

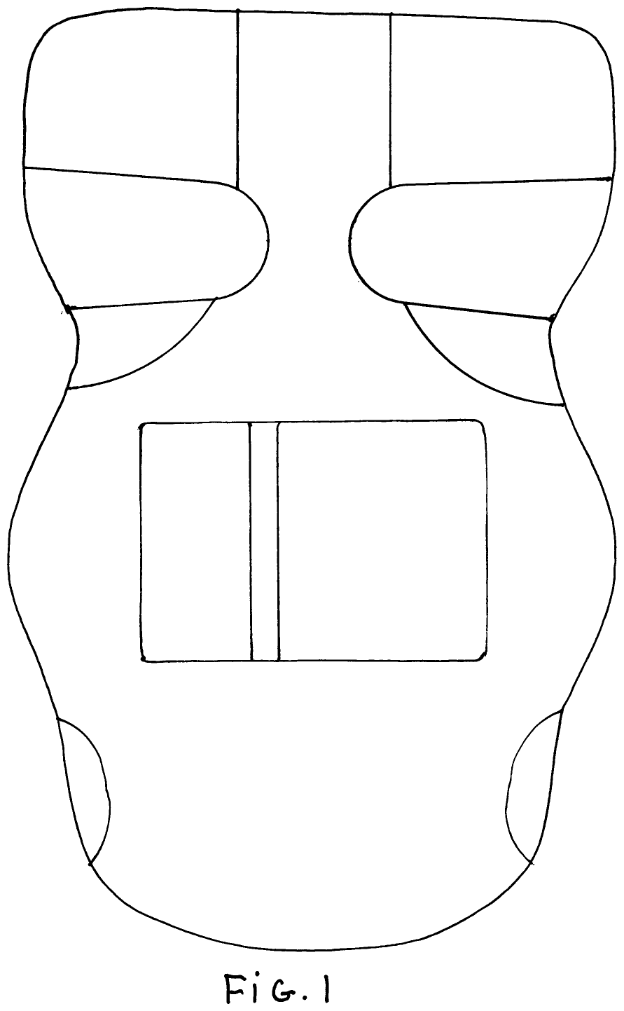

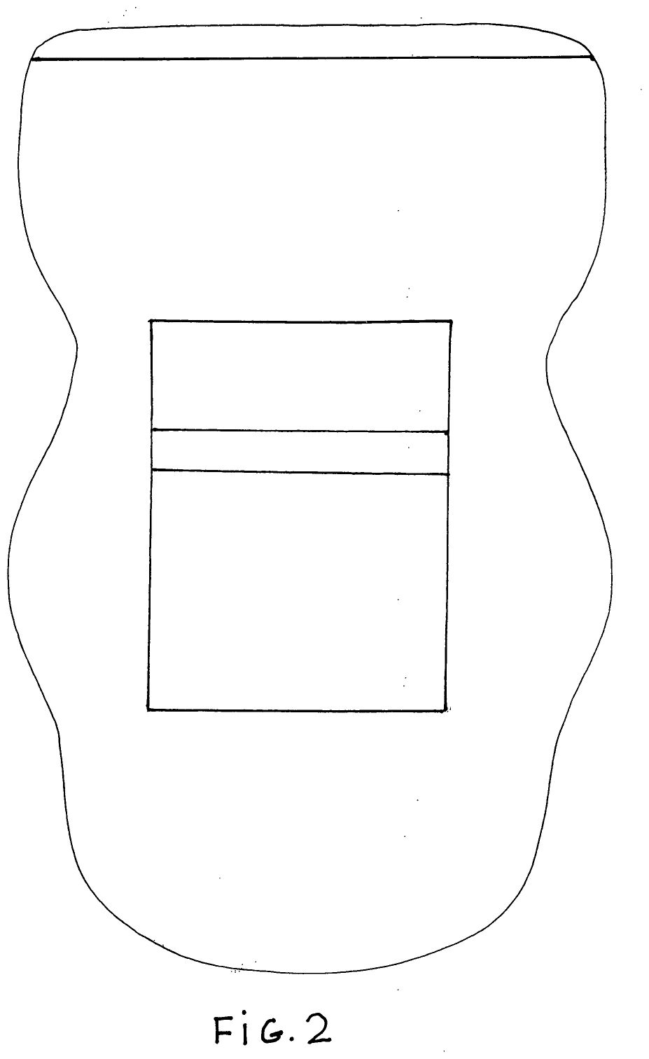

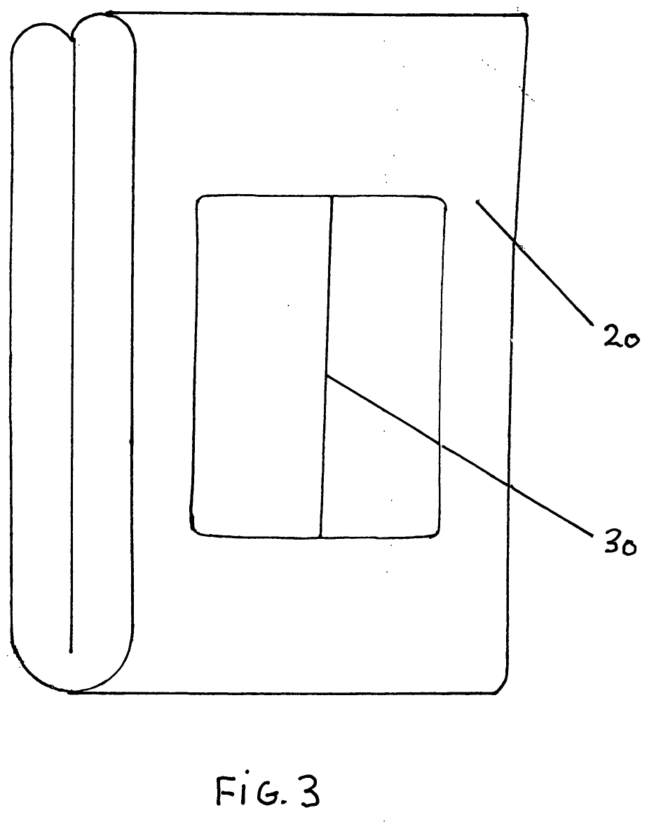

The invention is presented in term of its preferred embodiment herein depicted within FIG. 1 through 3 is shown and described for the purpose of clarity and disclosure and not intended to be limiting of the invention.

The present invention Diapers 20 with wipes 30 is a product modification the concept of Diapers 20 with wipes 30 is a method of packaging two diapering product into one and include the placement of a removable foil packet of wet wipes 30 in a packet/pouch on the front or back of the Diaper 20. The packet would be waterproof to keep the wipes 30 from drying out and would contain four or five wipes.

The diapers 20 with wipes 30 would be packaged together in conventional diaper packaging.

Diapers 20 with Wipes 30 would be appealing to many parents and caregivers because. They would save considerable time when changing a baby, everything needed for a diaper change would be within easy reach and would always be available when needed. The diapers 20 would be offered in full range of sizes for infants and toddlers. In addition, the diapers 20 with wipe 30 concept could be incorporated into disposable diapers for incontinent and/or disable older children and adults.

Diapers 20 with wipes 30 is envision it would fulfill the need for a method of packaging common diapering products into one, to simplify process of changing a baby's diaper. The appealing feature of diapers 20 with wipes 30 would be convenience, time saving and potential cost saving, this concept could present. By packaging these diapers-changing products into one, busy parents could save time and possibly even money. In the home parents would not have to search for baby's wipes just as the baby was ready for a diaper change. Parents would maintain their attention focus on the safety of the baby instead of searching for a container of wipes. This is particularly important with older babies and active toddlers who could easily roll off a changing table or other surfaces.

For busy parents on the go, Diapers 20 with wipes 30 concept would be particularly appealing. Parents could simply put one or more Diaper 20 with Wipes 30 in the baby's bag. The wipes 30 would be ready for a diaper 20 change and could even be pre-warmed while the diaper 20 is worn by the baby. The wipes 30 would be available even if parent/caregivers did not have access to a diaper bag.

Parents would be provided peace of mind knowing they would never run out of wipes, with “Diaper 20 with Wipes 30”, they would be available with the diaper 20. The Diaper 20 with Wipes 30 could be used in daycare centers or other institutions that provide care for infants and toddlers and by individuals who provide baby-sitting services in their homes. The Diapers 20 with wipes 30 could also be extended to disposable diaper for incontinent children/adults and could be used in both home care, baby-sitting and institutions such as hospital and extended care faculties.

It is envision that Diapers 20 with Wipes 30 would work function as designed, serve the intended purpose and accomplish those objective desired and it will be clear to any ordinary person skilled in the art to which the invention pertains, that there are many other embodiments possibility of the invention without department from the spirit and the cope of the present invention.

The potential exists for varying the production of Diaper 20 with Wipes 30 in ways which could make them more appealing to a wide range of users. This could includes offering them in a full range of diaper sizes for babies and toddlers, as well as older children and adults. The Wipes 30 could also be offered in different quantities, textures and formulations (scented, unscented, aloe added etc.).

Since “Diapers 20 with Wipes 30” would represent a modification of an existing product they would be readily producible. The manufacture of the diaper portion of this product would strongly parallel the production of the conventional disposal diaper 20 with the addition of the removable wet wipes 30 packet added to the front or back as the major modification. Disposable diapers are generally produced of cellulosic fiber papers and/or absorbent cotton and gel materials. Standard elastic would be used for the waist and legs. Velcro could be used at the sides.

The Diapers 20 with Wipes 30 could be package in imprinted polyvinyl chloride plastic pouch that would be heated sealed to protect the content. A perforated top could be added to provide easy access to the wet wipes 30 on the diaper 20. Corrugated cardboard shipping container would then be used to whole a quantity of individually package products for facilitate shipment and storage.

The embodiment suggested a concept that would combine two essential diapering products into one. The Diapers 20 with Wipes 30 would be a conventional disposable diaper with a removable packet containing wet wipes 30 on the front or back of each diaper 20. When it is time for a diaper change parents or caregivers could simply remove and open the packet of wet wipes 30 and use them to clean the baby's bottom. Combining the diapering products into one would save busy parents time. The wet wipes 30 would be handy a ready for use with the diaper 20. This would increase safety of the diaper changing procedure. The Parent would not have to search or struggle for wipes 30, while changing the baby on the changing table.

Diapers 20 with Wipes 30 with the assurance that the needed products would be right there. For working mother and fathers who must provide diapers and wipes for daycare or baby-sitters. These Diapers 20 with Wipes 30 would be especially convenient. They would be great for everyday outings for trips to the zoo, amusement parks etc. In addition, to marketing this product to home consumers, consideration could be given to marketing a product. Such as Diapers 20 with Wipes 30 to institutional facilities, daycare centers and hospitals. This packaging concept would also save valuable space in a baby's bag and lighten the load. The preferred embodiment of the present invention has been presented and described for the purpose of illustration, they not intended to be exhaustive or to limit the invention. It will be clear to those killed in the art that other changes in forms and various embodiment and example that may be perform in many modifications. The embodiment is contemplated thereby and are intended to be covered by the following claims.

What is claimed is:

1. A removable packet of wet wipes configured on the front or back of the diaper comprises of a diaper with an attachment to the said diaper that would be placed on the front or back of said diaper.

2. The packet of wet wipes of claim 1 wherein said wet wipes place on front of diaper.

3. The packet of wet wipes of claim 2 wherein wet wipes place on the back of said diaper.
